(19) United States
(12) Patent Application Publication
(10) Pub. No.: US 2015/0174325 A1
(43) Pub. Date: Jun. 25, 2015

(54) INJECTOR DEVICE WITH MECHANISM FOR PREVENTING ACCIDENTAL ACTIVATION

(71) Applicant: OVAL MEDICAL TECHNOLOGIES LIMITED, Cambridge (GB)

(72) Inventors: Matthew Young, Cambridge (GB); Ralph Lamble, Cambridge (GB)

(21) Appl. No.: 14/413,592

(22) PCT Filed: Jul. 9, 2013
(86) PCT No.: PCT/GB2013/051802

§ 371 Date: Jan. 8, 2015

(30) Foreign Application Priority Data
Jul. 9, 2012 (GB) 1212190.1

(51) Int. Cl. A61M 5/20; A61M 5/32

(52) U.S. Cl. CPC A61M 5/2033; A61M 5/3202; A61M 2005/2073

(57) ABSTRACT

There is provided an injector device comprising a housing (26), a drug container (10) positioned in the housing; a drug delivery mechanism associated with the drug container, the drug delivery mechanism comprising a stored energy source; an activation member (18) coupled to the housing, the activation member configured to contact the injection site in use and movable relative to the housing to an activation position to activate the drug delivery mechanism; and a removable cap (134) coupled to the activation member and to the housing, wherein in a first position a first portion of the housing engages the activation member to prevent the activation member from moving into the activation position, and wherein a portion of the cap engages the housing to retain the first portion of the housing in the first position such that the activation member is prevented from moving into the activation position until the cap is uncoupled from the activation member.

### FIELD OF THE INVENTION

The present invention relates to injector devices, such as autoinjectors, that have an automatic mechanism that is triggered by pressing the device against the injection site. In particular the invention relates to mechanisms for preventing accidental triggering of the automatic mechanism prior to intended use.

### BACKGROUND TO THE INVENTION

Prefilled injector devices allowing for self administration of drugs are becoming increasingly prevalent, as self administration has clear benefits in terms of cost to health care providers and well as improving patient convenience.

One type of prefilled injector device is an autoinjector, which includes an automatic delivery mechanism which, once activated, provides for automatic delivery of the drug. In autoinjectors, the delivery mechanism includes a stored energy source which, when released, drives the drug delivery mechanism and may also drive needle insertion.

Activation of the delivery mechanism can be achieved in a number of ways depending on the design of the device. However, there are clear benefits in terms of usability if the delivery mechanism is activated simply by pressing a front end of the device against the injection site. This removes the need to press any additional buttons or otherwise manipulate the device, which can be problematic for users suffering from disorders such as arthritis. Alternatively if the autoinjector includes an activation button or similar mechanism then an additional interlock unlatched by pressure on the front end of the device from the injection site can reduce the risk of accidental premature activation. The front end of the device is typically covered by a removable cap in order to keep the front end of the device that contacts the injection site clean prior to use. However, it is important to ensure that the delivery mechanism is not activated before the intended use of the device. Even though the front end of the device may be spring biased into an extended position, injector devices may be subject to high impact and vibration during transport and may be dropped during transport or by the end user prior to intended use. If the delivery mechanism is triggered, by vibration or impact forces before intended use, then the drug in the device is wasted and, with disposable, single use devices, the device itself is wasted. In addition, there is a potential needle stick injury risk if the device is activated before intended use. Another problem is that the user may be relying on a particular device to deliver the drug, not knowing that the device has already been activated. This is not good in any circumstances, but is dangerous when the drug is required in an emergency situation.

It is an object of the present invention to address the problem of unintended activation of automatic delivery mechanisms in injector devices.

### SUMMARY OF THE INVENTION

The invention is defined in the appended claims to which reference should be made.

In one aspect there is provided an injector device comprising: a housing; a drug container positioned in the housing; a drug delivery mechanism associated with the drug container, the drug delivery mechanism comprising a stored energy source; an activation member coupled to the housing, the activation member configured to contact the injection site in use and movable relative to the housing to an activation position to activate the drug delivery mechanism; and a removable cap coupled to the activation member such that the activation member is prevented from moving into the activation position until the cap is uncoupled from the activation member.

Using a removable cap to prevent relative movement between the activation member and the housing means that the delivery mechanism can only be operated after the cap has been removed from the device. A cap is typically secured to the device to maintain the portion of the device that contacts the injection site clean prior to use. As an alternative, or in addition, the cap may function to remove a needle shield which keeps the needle sterile prior to use and which may also seal the end of the needle to prevent premature loss of drug. The cap is secured to the device or is retained by secondary packaging in such a way that it will not easily come away from the device during transport and handling.

The cap may be coupled to the housing. The cap may abut or engage the housing to prevent the cap and activation member together moving relative to the housing. As an alternative, or in addition, the cap may be coupled to the drug container to prevent the cap and activation member moving together relative to the housing. The cap may be coupled to a needle shield that covers a needle coupled to the drug container.

The activation member may move telescopically with respect to the housing. The activation member may comprise a generally cylindrical member and may have a substantially closed front end except for an aperture allowing for passage of a needle therethrough. The front end then provides a front surface for contacting an injection site. A rear end of the activation member may engage with one or more parts of the delivery mechanism or with a locking arrangement for the delivery mechanism.

In some embodiments, in a first position, a portion of the cap, or an intermediate component coupled to the cap, may engage the activation member to prevent the activation member from moving into the activation position, and a portion of the housing may engage the cap to retain the cap in the first position.

The cap may be coupled to an exterior surface of the activation member. For example, the cap may be coupled to the activation member by a helical threaded engagement. The cap may also (or alternatively) be coupled to the housing by a helical threaded engagement. The helical threads on the activation member and on the housing may have the same helix angle.

The cap may engage a lug, recess or aperture on the activation member to prevent the activation member from moving into the activation position. The cap may comprise a flexible arm that engages with the lug, recess or aperture on the activation member. For example, the cap may be pushed onto the activation member in a longitudinal direction and may comprise flexible arms that flex in a direction non-parallel with the longitudinal direction to engage with the lug recess or aperture on the activation member. The flexible arm may be retained in engagement with the lug, recess or aperture on the activation member by a portion of the housing. When the cap is moved away from the housing in a longitudinal direction, the portion of the housing is removed from engagement with the flexible arm, allowing the flexible arm to be released from engagement with the lug, recess or aperture in the activation member.

The housing may comprise a cam surface to assist in decoupling of the cap from the activation member as the cap is removed from the housing. For example, the cap may include a lug that engages a lug or recess in the activation member and a cam surface on the housing may be provided adjacent to the lug or recess to urge the cap out of engagement with the lug or recess on the activation member as it is removed from the housing.

Alternatively, or in addition, in some embodiments, the cap may be coupled to an interior surface of the activation member. The cap may be directly coupled to an interior surface of the activation member or may be coupled to the interior surface of the activation member by one or more intermediate components. For example, the intermediate component may be a removable needle shield provided to maintain a needle in a sterile condition. Alternatively, the intermediate component may be an additional element coupled to a removable needle shield, the removable needle shield being coupled to the cap and to the drug container.

The intermediate component may comprise a resilient element that is deformed by the cap to engage the activation member to thereby prevent the activation member from moving into the activation position. Movement of the cap relative to the activation member may then release the resilient element from engagement with activation member allowing the activation member to move to the activation position. For example, the removable needle shield may comprise a resilient element that is deformed by the cap when the cap is fully engaged with the housing to engage a rear facing surface of the activation member if the activation member moves towards the activation position. Initial movement of the cap away from the housing releases the resilient element to allow it to return to a configuration in which it can pass through an aperture in a front surface of the activation member.

Alternatively, the intermediate component may have a flexible arm that engages the activation member in an unstressed state, configured such that movement of the cap away from the housing deforms the flexible arm to decouple the flexible arm from the activation member.

The cap may be coupled to the activation member by engagement of a thread on the cap with a thread on an internal surface of the activation member or on an intermediate element positioned inside the activation member and between the cap and the activation member. The engagement of the threads on the activation member and the cap may require less than a full turn of rotation to move from a fully engaged position to a fully disengaged position. Alternatively they may require more than one full rotation to move from a fully engaged position to a fully disengaged position.

The activation member may comprise a skin contact surface configured to contact the injection site in use, the skin contact surface including at least one aperture, and the cap may comprise an engagement element configured to be received in the at least one aperture such that relative rotation between the cap and the activation member causes the engagement element to engage the activation member to prevent the activation member from moving into the activation position. This arrangement is similar to a bayonet type fitting as an initial relative rotation is required to disengage the cap from the activation member but thereafter they can be separated by a translational movement.

In another embodiment, in a first position a first portion of the housing engages the activation member to prevent the activation member from moving into the activation position, and a portion of the cap engages the housing to retain the first portion of the housing in the first position. The first portion of the housing may be flexible (and may be resilient) and the cap may engage the housing to prevent the first portion of the housing moving out of a first position engaging the activation member, and may deflect the first portion of the housing into the first position, wherein on removal of the cap, the first portion of housing is able to move out of the first position to allow the activation member to move to the activation position. In the first position the first portion of the housing may engage a slot or recess in the activation member. The activation member may include a cam surface configured to allow the activation member to move past the first portion of the housing after the cap has been removed from the housing.

It should be clear that features of the invention described in relation to one aspect may be applied to other aspects of the invention, and that features described in different embodiments of the invention may be used in combination with one another.

### BRIEF DESCRIPTION OF THE DRAWINGS

Embodiments of the invention will now be described in detail, by way of example only, with reference to the accompanying drawings, in which:

FIG. 1 is a perspective, cross-sectional view of an autoinjector configured to be activated by pressing a front mounted activation member against the injection site;

FIG. 2 shows the mechanism by which the autoinjector of FIG. 1 is triggered;

FIG. 3 is a perspective view of an autoinjector in accordance with a first embodiment of the invention;

FIG. 4 is a perspective view of a cap for use in the embodiment of FIG. 3;

FIG. 5 is a cross-sectional view of the first embodiment, with the cap engaged with the housing and the activation member;

FIG. 6 is a perspective view of a second embodiment of the invention, with the cap in a partially removed position;

FIG. 7 is a perspective view of the second embodiment with the cap on, but with a portion of the housing removed for clarity;

FIG. 8 is a detailed view of the engagement of the cap, activation member and housing during removal of the cap;

FIG. 9 is a detailed view of the cap activation member and housing during cap removal, immediately after the view shown in FIG. 8;

FIG. 10 is a cross-section of a front end of an autoinjector in accordance with the third embodiment of the invention;

FIG. 11 is a cross-sectional view of the third embodiment as shown in FIG. 10, but with the cap removed;

FIG. 12 is a perspective view of the front end of an autoinjector in accordance with a fourth embodiment of the invention with the cap removed;

FIG. 13 is a cross-sectional view of the embodiment of FIG. 12, with the cap engaged;

FIG. 14 is a cross-sectional view of the fourth embodiment during removal of the cap;

FIG. 15 is a cross-sectional view of a front end of an autoinjector in accordance with a fifth aspect of the invention;

FIG. 16 is a perspective view of the intermediate component shown in FIG. 15;

FIG. 17 is a perspective, cross-sectional view of the cap and intermediate component assembled together;

FIG. 18 corresponds to the view shown in FIG. 15, with the cap partially removed from the housing;

FIG. 19 shows the view of FIG. 18 with the cap further removed from the housing, with the intermediate component deformed;

FIG. 20 is a cross-sectional view of the embodiment of FIG. 15 with the cap fully removed from the housing;

FIG. 21 is a perspective view of an alternative intermediate component incorporating elements for gripping a removable needle shield;

FIG. 22 is a cross-sectional view of the front end of an autoinjector incorporating the intermediate component shown in FIG. 21;

FIG. 23 illustrates the embodiment shown in FIG. 22, during removal of the cap from the housing;

FIG. 24 is a cross-sectional view through the front end of an autoinjector in accordance with a seventh embodiment of the invention;

FIG. 25 is a perspective, cross-sectional view of the cap and intermediate component shown in FIG. 24;

FIG. 26 is a cross-sectional view of the embodiment shown in FIG. 24, with the intermediate component in a tensioned state;

FIG. 27 is a cross-sectional view of the embodiment of FIG. 24 during removal of the cap from the housing;

FIG. 28 is a perspective view of the front end of an autoinjector in accordance with an eighth embodiment of the invention, with the cap removed;

FIG. 29 is a perspective, cross-sectional view of a cap in accordance with the eighth embodiment;

FIG. 30 is the perspective, cross-sectional view of the eighth embodiment showing the cap engaged with the activation member;

FIG. 31 is a perspective view of the front end of an autoinjector in accordance with a ninth embodiment of the invention, with the cap removed;

FIG. 32 is a perspective, cross-sectional view of a cap in accordance with the ninth embodiment of the invention;

FIG. 33 is a perspective, cross-sectional view of the ninth embodiment of the invention with the cap engaged with the activation member;

FIG. 34 is a perspective view of the front end of an autoinjector in accordance with a tenth embodiment of the invention;

FIG. 35 is a perspective, cross-sectional view of a cap for use with the tenth embodiment of the invention;

FIG. 36 is a perspective, cross-sectional view of the tenth embodiment of the invention with the cap engaged with the activation member;

FIG. 37 illustrates the cap of FIG. 36 rotated to a position where the cap can be removed by a pull-off motion;

FIG. 38 is a cross-sectional view of the tenth embodiment of the invention during removal of the cap;

FIG. 39 is a perspective view of an autoinjector in accordance with an eleventh embodiment of the invention;

FIG. 40 is a perspective, cross-sectional view of the embodiment shown in FIG. 39;

FIG. 41 is a perspective view of the cap shown in FIG. 40;

FIG. 42 is a perspective, cross-sectional view of the activation member shown in FIG. 40;

FIG. 43 is a perspective, cross-sectional view of the embodiment shown in FIG. 40, with the activation member moving into the housing; and

FIG. 44 shows the embodiment of FIG. 43, with the activation member moved to the activation position.

### DETAILED DESCRIPTION

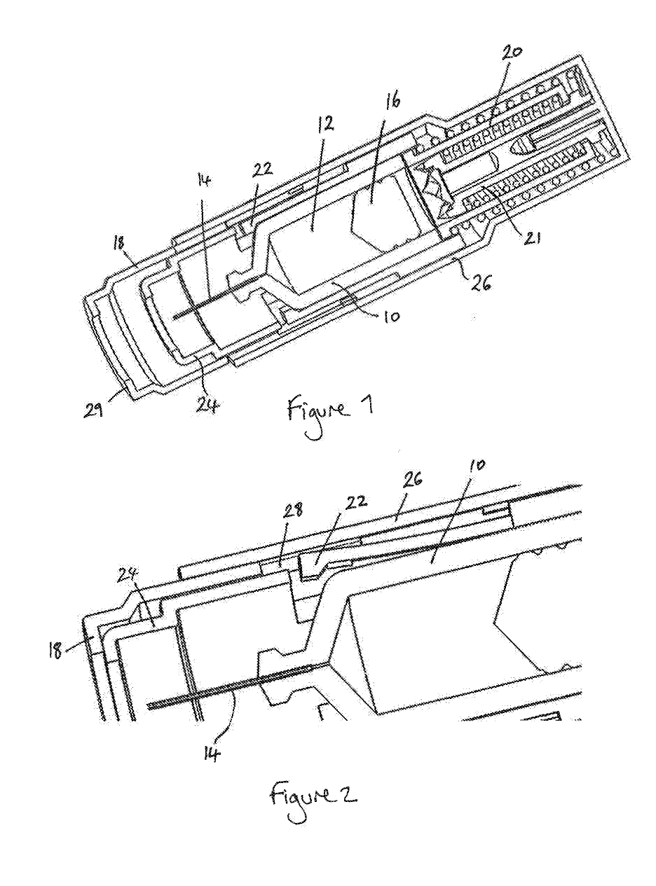

FIG. 1 is a perspective, cross-sectional view of an autoinjector configured to be activated by pressing an activation member 18 against an injection site. The autoinjector shown in FIG. 1 comprises a primary drug container 10 housing a drug 12. A plunger 16 is positioned within the drug container 10. In use, the plunger 16 is driven through the drug container to expel the drug 12 through a needle 14, which is fixed to the front end of the drug container 10.

The autoinjector has a housing 24, 26, which houses the drug container 10 as well as a drive mechanism 20. The activation member 18 is mounted to the front end of the housing 24, 26. As shown in FIG. 1 the housing 24, 26 comprises two parts, an outer housing 26 and an inner housing portion 24 that is fixed to the outer housing 26. However, it should be clear that these may be formed as a single component.

The drive mechanism 20 as shown in FIG. 1 comprises two springs held in a compressed condition prior to use. A first, outer spring is used to drive the drug container 10 forward through the housing to a needle insertion position, in which the front end of the needle extends beyond the activation member 18. A second spring is used to drive a drive rod 21, which engages the plunger 16, to drive the plunger through the drug container 10 to expel the drug through the needle 14. The drug container is retained against the force of the drive mechanism 20 by latching arms 22, which engage the front end of the drug container 10 against the force of the springs in the drive mechanism.

As shown in FIG. 2, when the activation member 18 is pressed against the injection site it moves backwards into the housing 26 and a window 28 in the activation member 18 is moved back to a position adjacent to the front end of retaining arms 22. In this position, the arms 22 are able to flex outwardly into the windows 28 allowing the drug container to move forward through the housing to a needle insertion position. The drug container 10 then moves forward through the housing until it is stopped by engagement with the housing 24, after which the plunger 16 is driven through the drug container 10 to expel the drug. The operation of the drive assembly 20 will not be described in detail in this specification, as any suitable drive assembly may be used, incorporating one more springs, or any suitable type of stored energy. A detailed description of a drive assembly of the type shown in FIGS. 1 and 2 can be found in WO2012/073035, the contents of which are incorporated herein by reference.

Although not shown in FIGS. 1 and 2, a needle shield element may be provided to cover the needle in order to maintain the needle 14 in a sterile condition prior to use. A cap is also typically provided over the front end of the housing to maintain the activation member, and in particular the surface 29 of the activation member 18 that contacts the injection site, clean prior to use. The cap may also assist in the removal of the needle shield element by the user prior to the injection process.

However, with a cap over the front end of the housing 26, it is still possible for the activation member 18 to move to an activation position, i.e. the position where the latching arms 22 can move into windows 28 in the activation member, unless some means is provided for preventing the activation member from moving to its activation position. A biasing spring is typically provided between the activation member and the housing 24 to bias the activation member away from the housing 24 (item 39, as shown in FIG. 5) but this spring cannot be relied on to prevent activation of the device in all conditions.

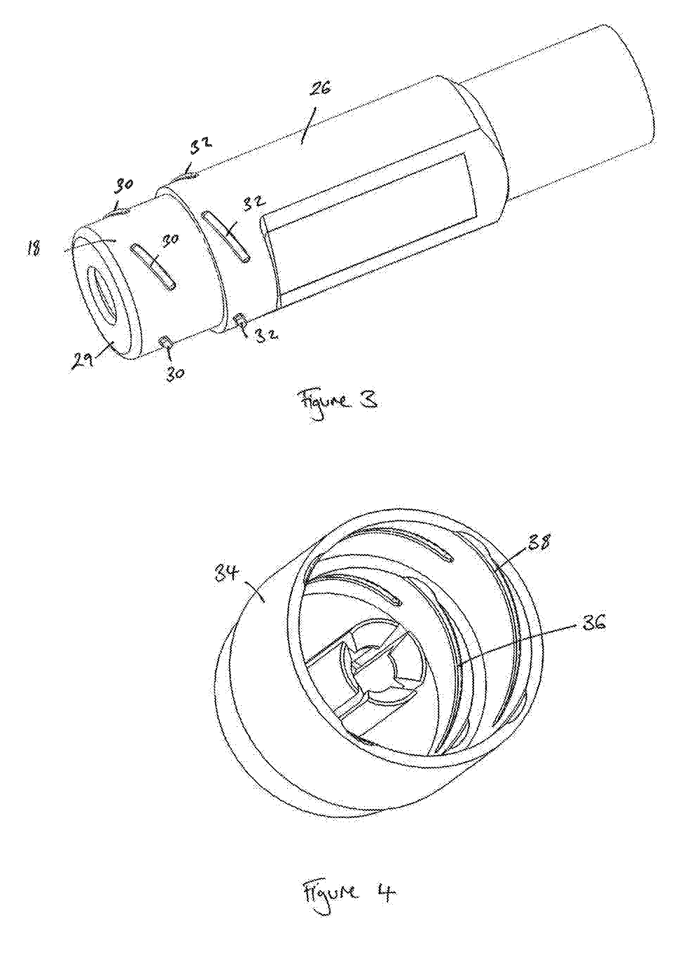

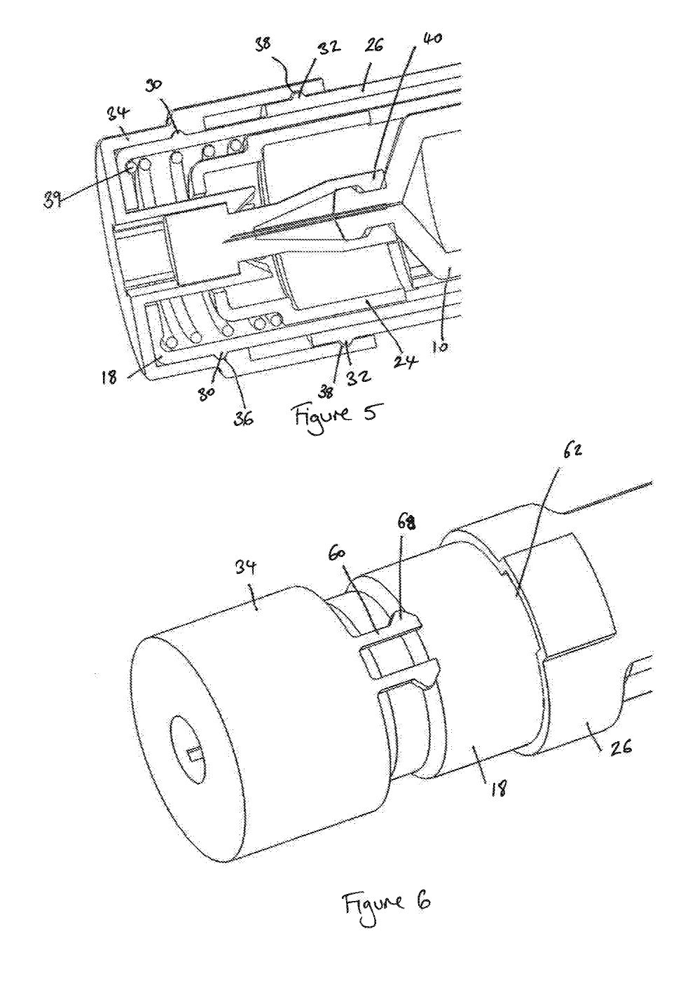

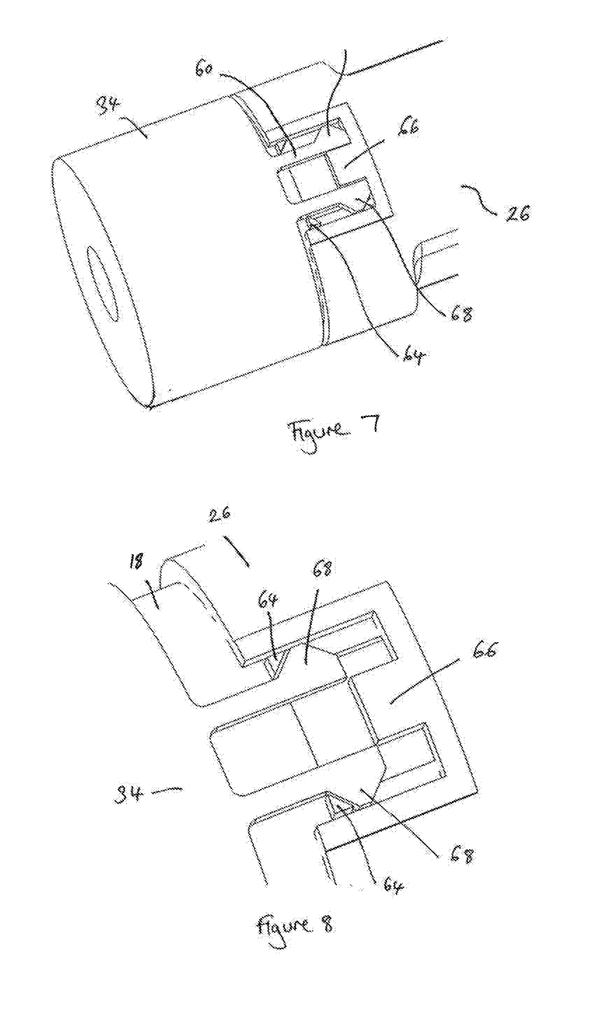

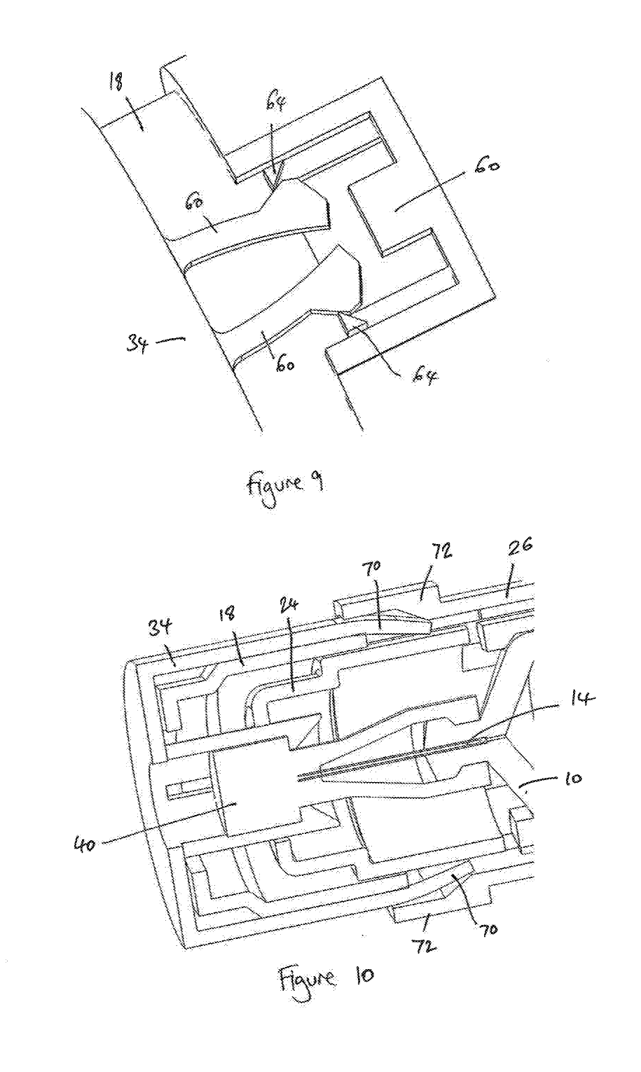

FIG. 3 is a perspective view of an autoinjector of the type shown in FIGS. 1 and 2 incorporating a mechanism for preventing the activation member 18 from moving to the activation position until a cap 34 (shown in FIGS. 4 and 5) is at least partially removed from the housing. In the embodiment shown in FIG. 3, thread elements 30 are formed on an outer surface of the activation member and corresponding thread elements 32 are formed on an outer surface of the housing 26. These thread elements 30, 32 are configured to engage corresponding internal grooves 36, 38 on cap 34, as shown in FIGS. 4 and 5. The thread elements 30 have the same helix angle as thread elements 32. FIG. 4 is a perspective view of the cap 34, showing the internal grooves 36 that engage the threads 30 on the activation member, and the internal grooves 38 that engage the threads 32 on the housing 26. In the embodiment shown in FIGS. 3, 4 and 5, the thread elements comprise four, discontinuous thread elements, circumferentially spaced around the activation member 18 and housing 26. However, it should be clear that a single, continuous thread may be used, or any other number of thread elements.

FIG. 5 is a cross-section through the front end of the autoinjector of FIG. 3 with the cap 34 of FIG. 4 engaged and an associated needle shield 40. The cap 34 is screwed onto the front end of the autoinjector and helical grooves 36 engage with the threads 30 on the activation member 18, and helical grooves 38 engage with the threads 32 on the housing. The cap 34 can be screwed onto the activation member 18 and housing 26 during device assembly. The cap 34 is then effectively braced between the activation member 18 and the housing 26 and so prevents movement of the activation member 18 relative to the housing 26 thereby preventing activation of the device prior to cap removal. A biasing spring 39 is also shown in FIG. 5, which ensures that the activation member is biased into an extended position before and after use. However, the biasing spring 39 alone is not sufficient to prevent accidental activation of the device.

FIGS. 6, 7, 8 and 9 illustrate a second embodiment in accordance with the invention, in which the cap engages with protrusions 64 on the activation member 18, and with the housing 26, to prevent movement of the activation member to the activation position. FIG. 6 is a perspective view of the front end of an autoinjector in accordance with this second embodiment. The cap 34 comprises flexible arms 60 which, when assembled to the autoinjector, extend into and are retained in a receiving recess 62 in the housing 26. FIG. 7 illustrates the embodiment of FIG. 6 with a portion of the housing 26 removed for clarity. It can be seen in FIG. 7 that the flexible arms 60 include enlarged head portions 68 which prevent the activation member, and specifically projection 64, from moving beyond them. The flexible arms 60 can flex to allow the enlarged head portions 68 to move past the projection 64 but are prevented from flexing when engaged with portion 66 of the housing 26. If the activation member 18 is moved backwards prior to use, lugs 64 will come into engagement with enlarged head portions 68 of the flexible arms 60 and are prevented from moving further back into the housing 26. This prevents inadvertent activation of the device.

Removal of the cap 34 from the housing 26 is illustrated in FIGS. 8 and 9. As the cap is pulled off the autoinjector, in a first stage the enlarged head portions 68 are moved to a position where they are free of the portion 66 of the housing. This is shown in FIG. 8. Further pulling of the cap results in inward deflection of the legs 60, as shown in FIG. 9, allowing the enlarged head portions to move past the lugs 64, thereby releasing the cap 34. Once the cap 34 has been removed, the activation member 18 is free to move backward to an activation position, allowing the autoinjector to be operated. The cap 34 and flexible arms 60 can be formed by injection moulding using any suitable material, such as polypropylene.

FIG. 10 is a perspective, cross-sectional view of a third embodiment of the invention. FIG. 10 shows the front end of an autoinjector of the type shown in FIG. 1, with a cap 34 and needle shield 40. In the embodiment of FIG. 10, the cap 34 includes flexible portions 70 which extend into an aperture formed in the activation member 18, and thereby prevent inward movement of the activation member to the activation position.

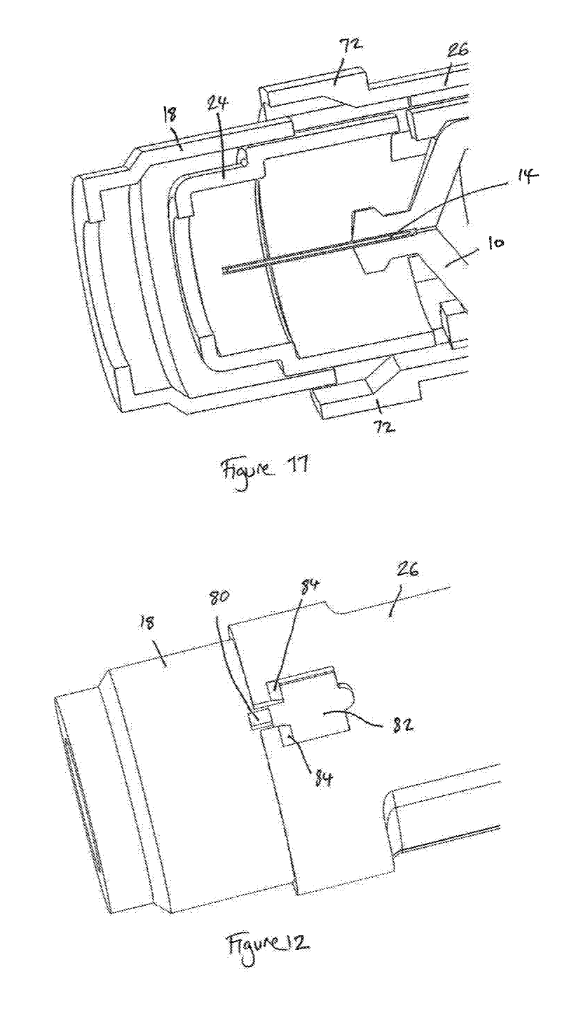

FIG. 11 shows the embodiment of FIG. 10 with the cap and needle shield removed. The housing 26 includes an enlarged portion 72 which defines a passageway through which the flexible portions 70 on the cap can pass and flex inwardly to engage a window in the activation member 18. To remove the cap 34, the cap is simply pulled off in a direction parallel to the axis of the needle 14. The cap may include a plurality of spaced apart flexible portions 70, spaced around the circumference of the cap or may include a single flexible portion. The cap and flexible portions can be formed by injection moulding using any suitable material, such as polypropylene.

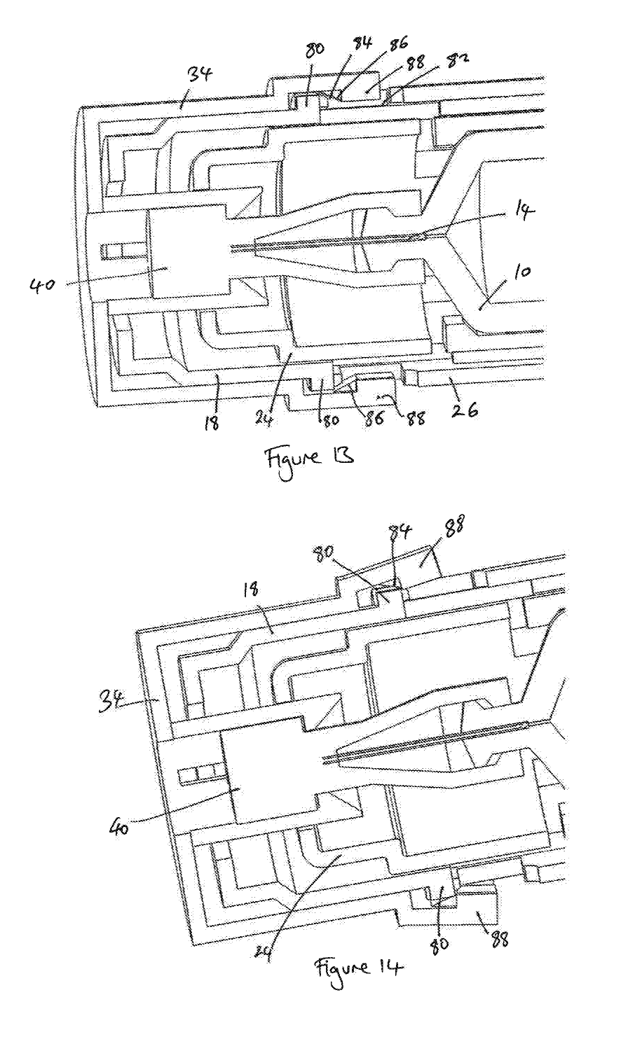

FIG. 12 is a perspective view of the front end of an autoinjector in accordance with a fourth embodiment of the invention. In the embodiment of FIG. 12, the outer housing 26 includes a window portion 82, into which a flexible portion of the cap 34 is received. The flexible portion of the cap in the window portion 82 engages a protrusion 80 on the activation member 18 to prevent movement of the activation member 18 into an activation position. FIGS. 13 and 14 are cross-sectional views of the embodiment of FIG. 12, illustrating the engagement of the cap 34 with the window 82. In order to facilitate removal of the cap, the housing includes cam surfaces 84 formed adjacent the window 82 that engage with the corresponding cam surfaces 86 on the interior surface of the cap. As the cap is pulled away from the housing, cam surfaces 84 engage with cam surface 86 and slide past one another, thereby urging the flexible portion of the cap 88 out of engagement with aperture 82 and past protrusion 80 formed on the activation member.

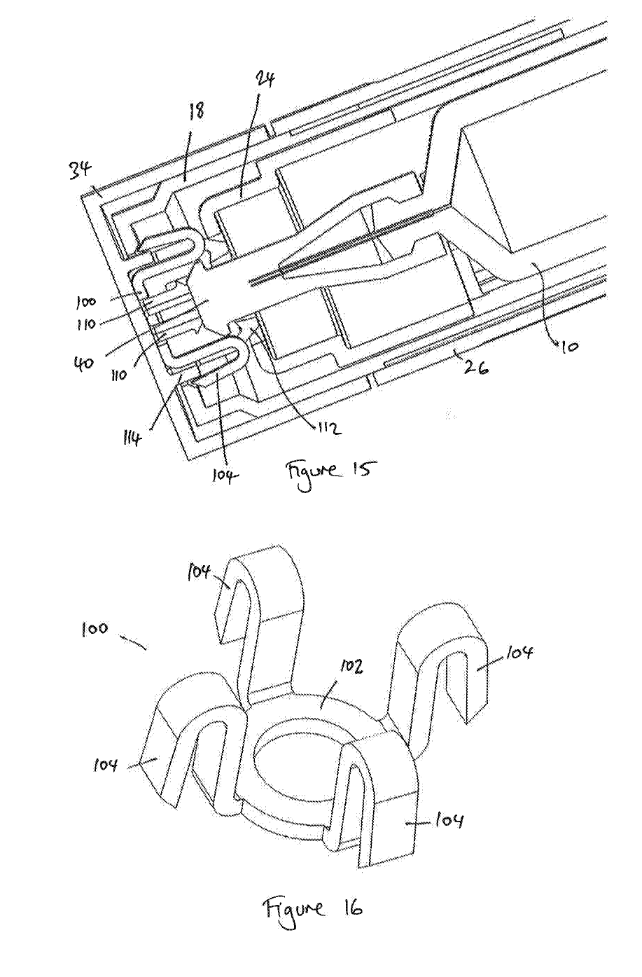

FIG. 15 is a cross-section through a front end of an autoinjector in accordance with a fifth embodiment of the invention. In the embodiment of FIG. 15 the activation member 18 is prevented from moving to the activation position by an intermediate component 100, coupled to the cap 34. The intermediate component is positioned substantially within an interior of the activation member 18. Movement of the activation member towards the activation position results in the front face of the activation member 18 abutting the intermediate component 100, which is itself constrained from movement by abutment with interior housing 24 and by latching element 110.

FIG. 16 is a perspective view of the intermediate component 100. As shown in FIG. 16, the intermediate component comprises a ring structure 102, from which four resilient, U-shaped legs 104 extend. The intermediate component 100 may be formed from any suitable resilient material, such as spring steel or a moulded plastic such as polypropylene, and may have a different number of legs.

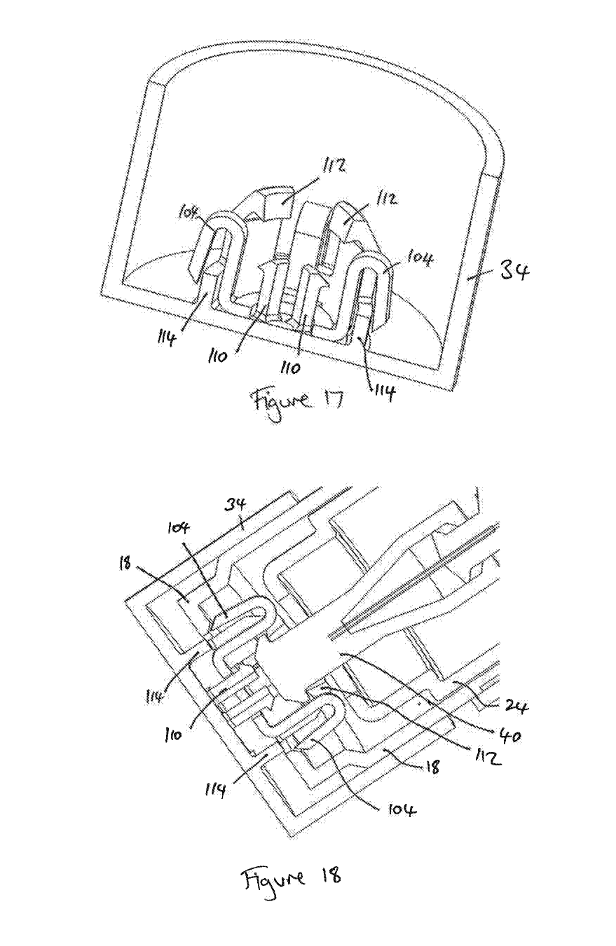

FIG. 17 is a perspective cross sectional view of the intermediate component 100 engaged with cap 34. The intermediate component 100 is pushed over and retained by latching element 110 formed inside the cap. The cap also includes resilient gripping arms 112, which are configured to grip needle shield 40 during cap removal, thereby removing the needle shield, and a retaining ring 114 that fits within the space formed underneath the U-shaped legs 104. As can be seen in FIG. 15, the retaining ring 114 prevents the legs 104 from radially compressing so that they cannot pass through the aperture formed in the front face 29 of the activation member 18.

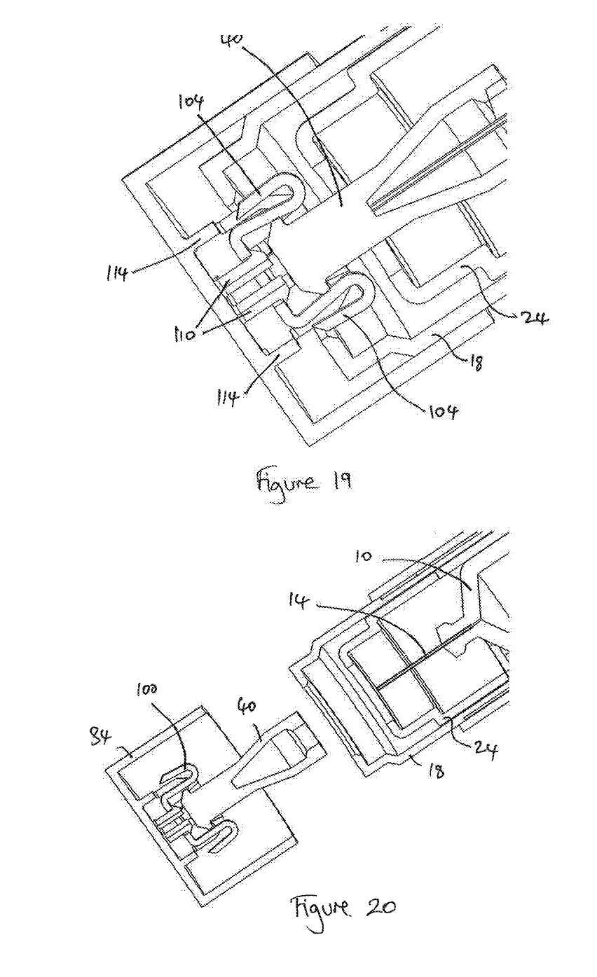

However, as the cap 34 is pulled away from the housing 26 the retaining ring 114 moves out of the space defined by U-shaped legs 104. This is shown in FIG. 18. As the cap 34 is further pulled away from the housing, the ring 102 abuts the latching element 110 and the legs 104 are then pulled down to engage the activation member 18. Angled surfaces on the activation member 18 and the ends of arms 104 engage one another such that movement of the intermediate component away from the housing causes the arms 104 to be radially compressed by the activation member, allowing the intermediate component to move through the aperture in the activation member 18. This is illustrated in FIG. 19. FIG. 20 shows the cap 34 and needle shield 40 fully removed from the activation member 18. The activation member 18 is then free to move into the activation position.

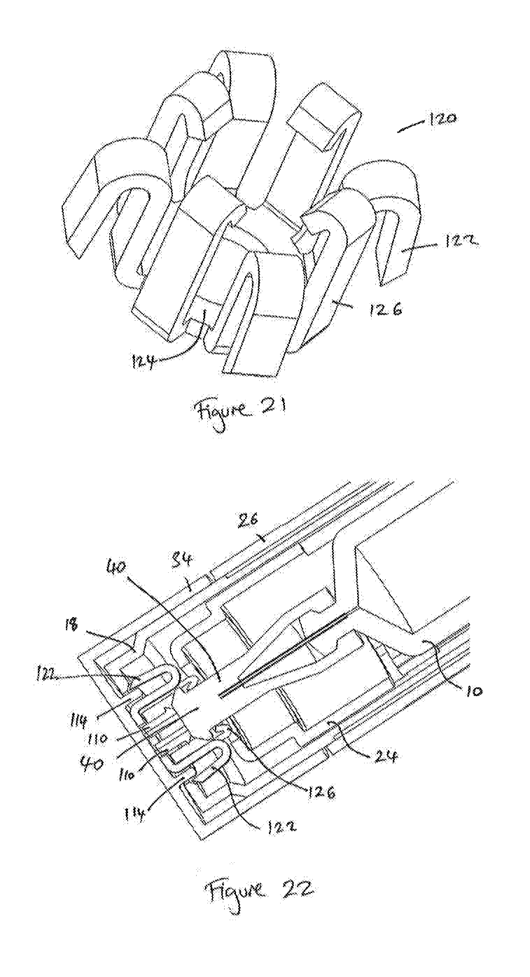

FIG. 21 is a perspective view of an alternative design for an intermediate component of the type shown in FIG. 16. In the embodiment of FIG. 21, in addition to resilient U-shaped legs 122 of the type described with reference to FIG. 16, the intermediate component 120 incorporates gripping arms 126 to grip the needle shield 40 extending from ring 124.

The operation of the embodiment of FIG. 21 is similar to that shown in FIGS. 16 to 20. FIG. 22 shows the cap and intermediate component 120 assembled to the activation member 18 and housing 26. The intermediate component is coupled to the cap by latching members 110. The legs 122 on the intermediate component prevent the activation member from moving back into the housing 26 to the activation position. The legs 122 are prevented from radially compressing to allow the activation member to move past the legs by retaining ring 114. Gripping arms 126 grip the needle shield 40.

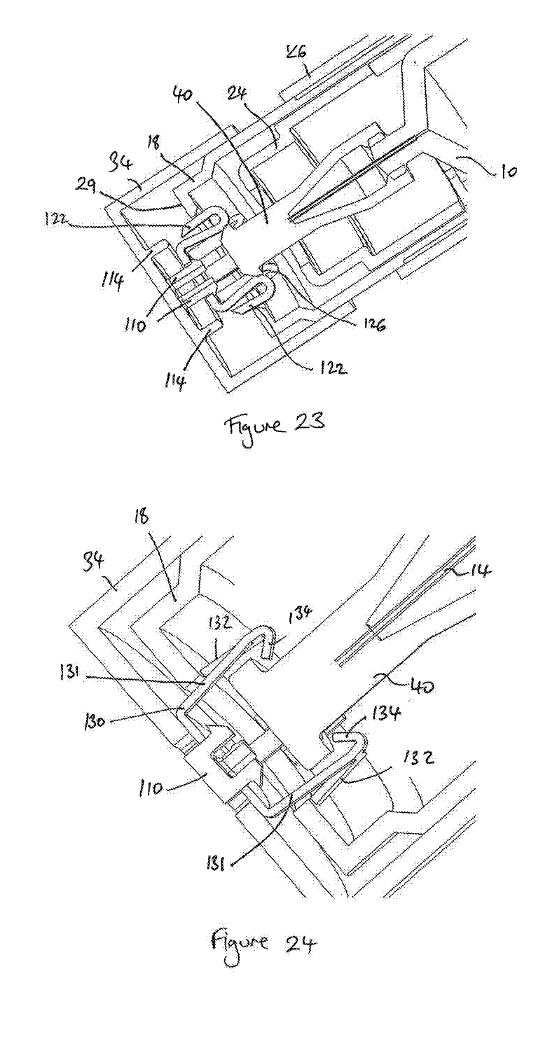

FIG. 23 shows the cap moved away from the housing to a position in which the legs 122 are clear of the retaining ring 114. The legs 122 have been radially compressed by the activation member 18 in the same manner as described with reference to FIG. 19, allowing the intermediate component 120 to pass through the aperture in the front face of the activation member 18. At the same time, gripping arms 126 have engaged the needle shield 40 and pulled it away from the drug container 10. As with the embodiment of FIGS. 16 to 20, the first part of the motion of the cap 34 away from the housing 26 allows the intermediate component 110 to move clear of the retaining ring 114 and in a second part of the motion of the cap 34 away from the housing 26 the intermediate component 120 is able to deform allowing it to move clear of the activation member 18.

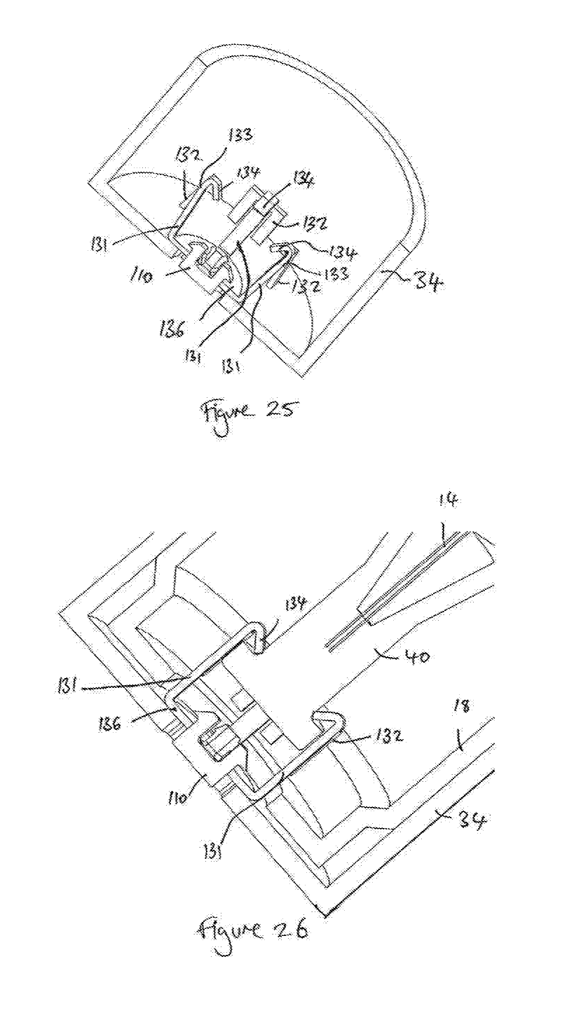

FIGS. 24 to 27 show a further alternative design for an intermediate component of the type, shown in FIG. 21. FIG. 24 is a partial perspective cross-sectional view of the front end of an autoinjector of the type shown in FIG. 1. The intermediate component 130 again provides both the function of locking the activation member 18 and gripping the needle shield 40. However, in the embodiment of FIGS. 24 to 27, the intermediate component 130 is deformed to allow it to pass through an aperture in the activation member by tensioning the intermediate component 130 between the needle shield 40 and the cap 34 as the cap is being removed from the housing. The intermediate component 130 is more clearly illustrated in FIG. 25, which is a cross-sectional view of the cap and intermediate component. The intermediate component 130 comprises four circumferentially spaced arms 131 on a ring structure 136. The intermediate component is retained on the cap by retaining latches 110. At the end of each arm remote from the ring structure is a gripping hook 134 for engaging the needle shield and a pair of locking fingers 132. The arms 131 include an elbow 133 so that they have a lower portion between the ring structure and the elbow, and an upper portion between the elbow and the gripping hook 134. The locking fingers 132 are attached to the upper portion and extend parallel to the upper portion so that their distal end cannot pass through the aperture in the front end of the activation member. Movement of the intermediate component 130 is limited by the latch element 110. The activation member 18 is therefore prevented from moving to the activation position by the locking fingers 132.

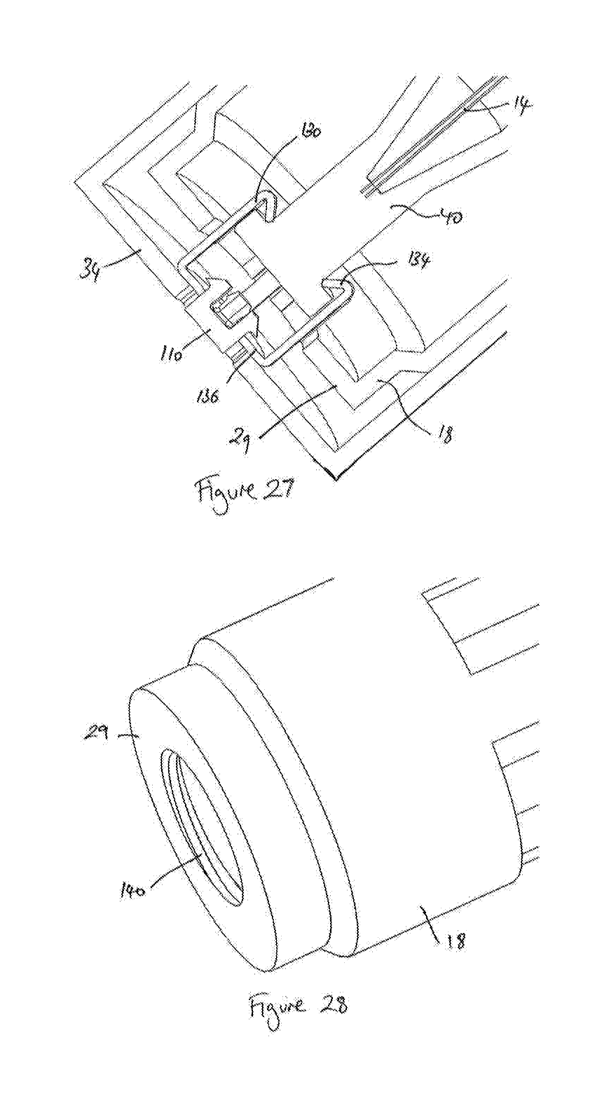

When the cap is pulled away from the housing, the gripping hooks 134 engage the needle shield 40. The needle shield 40 is sealed to the drug container 10 and so before the needle shield comes away from the drug container the intermediate component 130 is placed under tension by the movement of the cap 34 away from the housing 26. This tension causes the arms 131 to straighten at the elbow, which causes each locking finger 132 to move to a position parallel to the lower portion of the corresponding arm 131. This is shown in FIG. 26. In this tensioned state the locking fingers 132 can move through the aperture in the front face 29 of the activation member 18 and the intermediate component 130 and needle shield 40 can be completely removed from the rest of the device. This is shown in FIG. 27.

The embodiments of FIGS. 15 to 27 include an intermediate component between the cap 34 and the needle shield 40. It should be clear that it is possible to use the same type of mechanism to deform elements that engage an interior of an activation member, in which the deforming element are an integral part of the needle shield.

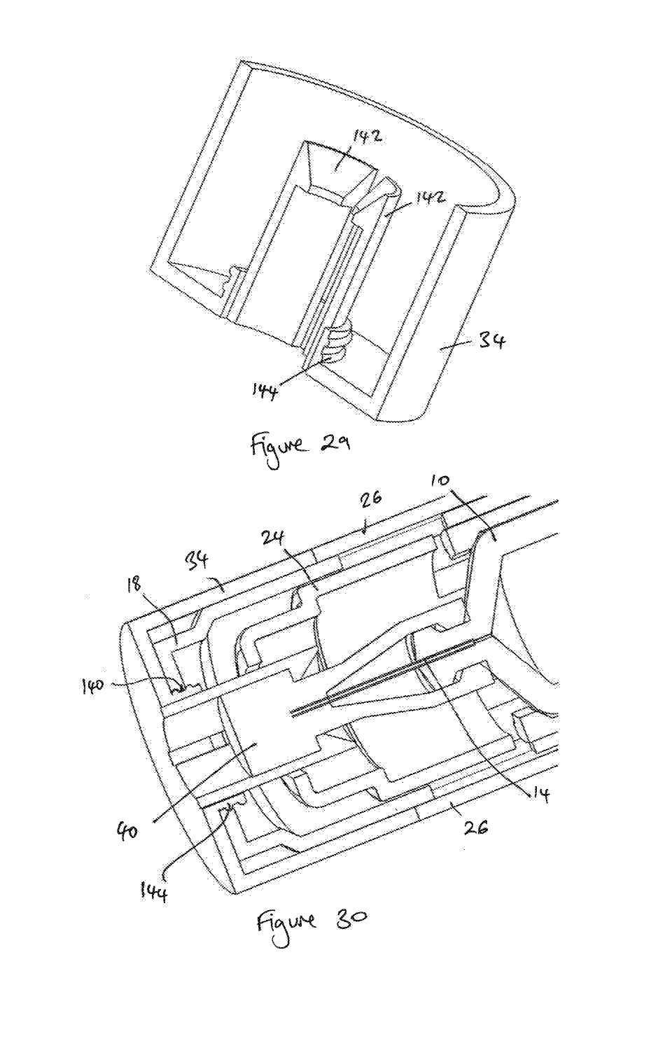

FIG. 28 is a perspective view of the front end of an activation member for use in an autoinjector in accordance with an eighth embodiment of the invention. In the embodiment of FIG. 28, a cap 34 (as shown in FIG. 29) is coupled to the activation member 18 by a screw thread engagement. The cap includes a thread 144 formed on an interior of the cap for engagement with a groove 140 formed on an interior surface of the activation member 18. The activation member is, as in the previous embodiments, a generally cylindrical shaped element with a front surface 29 configured to contact the injection site and has an aperture formed in the front surface, through which the needle passes. In the embodiment of FIG. 28 a bore of the aperture in the front face of the activation member has a helical groove 140 formed in it. FIG. 29 is a perspective cross sectional view of a cap 34 for engagement with the activation member 18 shown in FIG. 28. The cap 34 has a plurality of latching members 142 formed on an interior of the cap, for engagement with a needle shield 40, as shown in FIG. 30. A threaded shaft 144 is formed around the base of the latching members 142.

FIG. 30 shows the cap of FIG. 29 engaged with the activation member of FIG. 28. The cap 34 is screwed on the activation member 18 by engaging the threaded shaft 144 with the groove 140 and clips over and also engages the needle shield 40. The cap 34 then is effectively braced between the activation member 18 and the housing 26 and so prevents the activation member from moving to the activation position. To remove the cap 34, it is unscrewed from the activation member 18 using a twisting motion. As the cap is unscrewed from the activation member 18 it pulls the needle shield 40 from around the needle 14. Once the threaded shaft 144 is clear of the groove 140 the activation member 18 is free to move to the activation position.

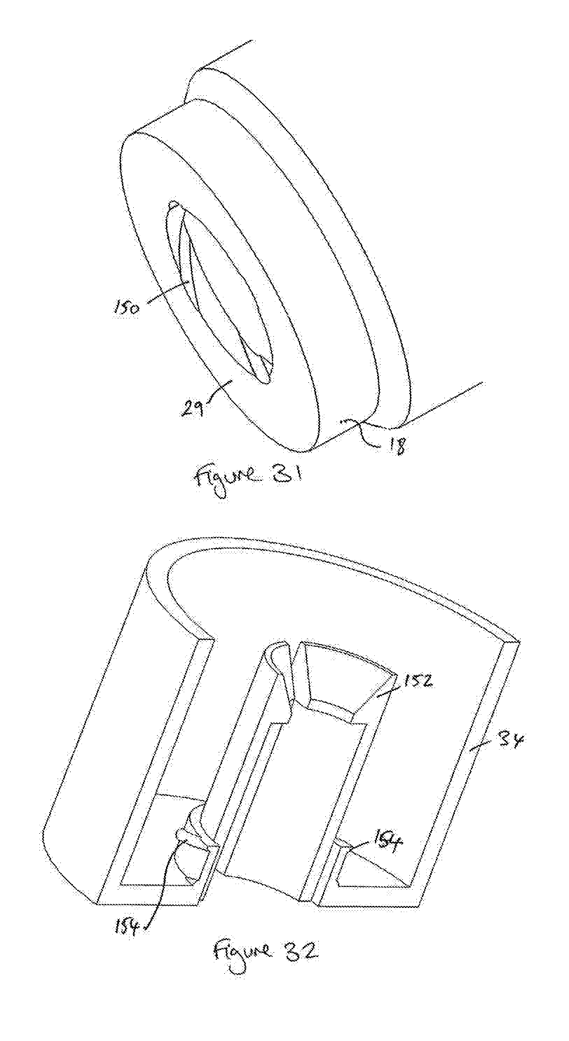

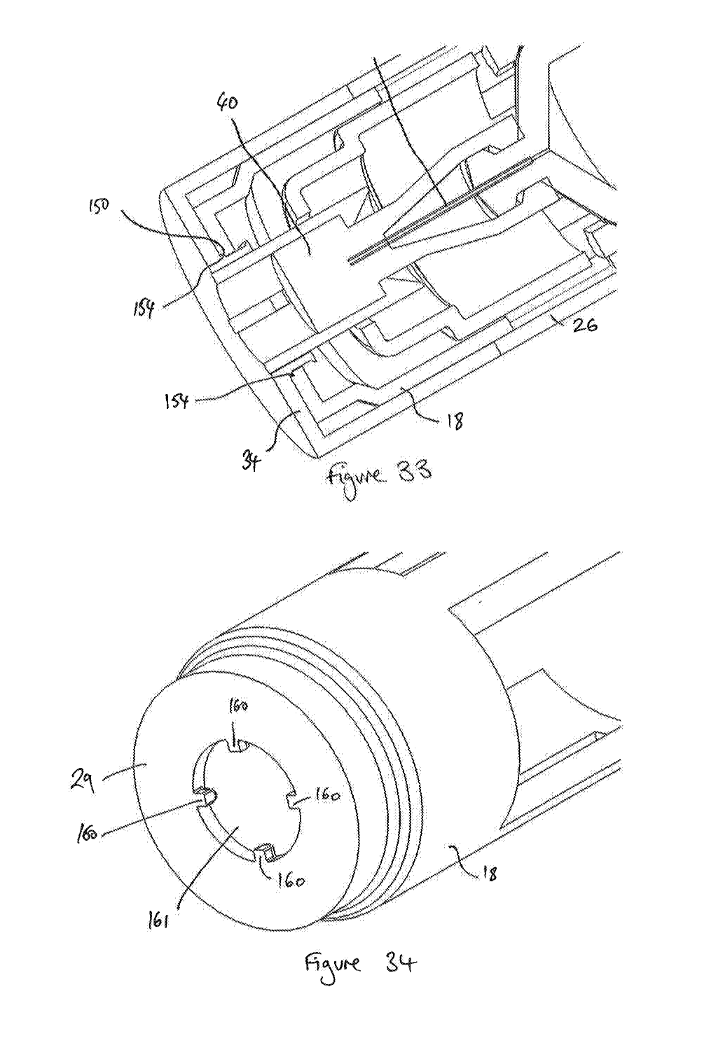

FIG. 31 is a perspective view of the front end of an activation member 18 in accordance with an alternative embodiment, similar to that shown in FIG. 28. In the embodiment of FIG. 31, instead of a single helical groove formed in the activation member 18, a plurality of separate grooves 150 are formed in the activation member. Each groove 150 engages with a corresponding bead or thread in the cap 34. FIG. 32 is a cross sectional perspective view of a cap 34 for engagement with the activation member 18 shown in FIG. 31. It is essentially the same as the cap 34 shown in FIG. 29 except for the design of the thread shaft 154. In the embodiments shown in FIGS. 31 and 32, the cap 34 can be disengaged and engaged with the activation member 18 by only a quarter turn between the two. FIG. 33 is a cross sectional view showing the cap of FIG. 32 engaged with the activation member of FIG. 31.

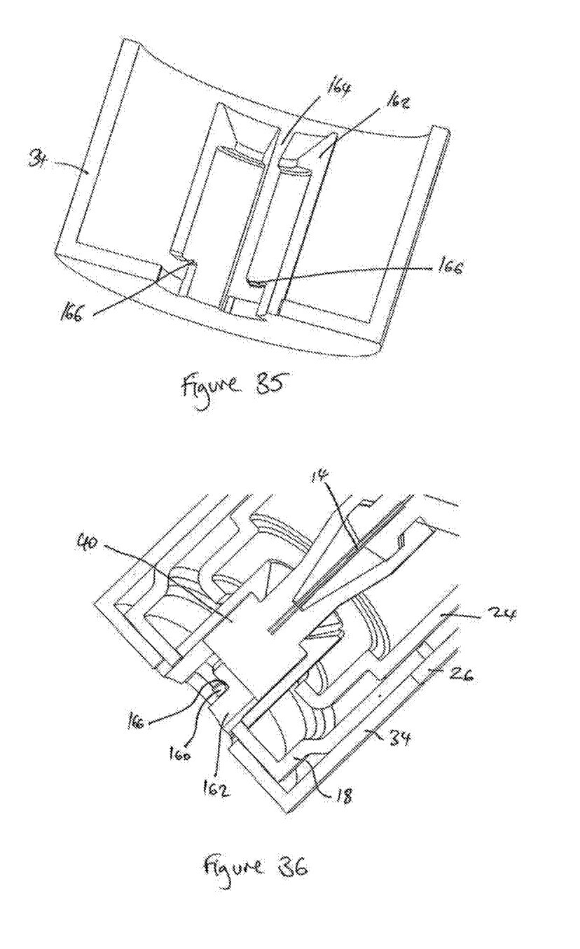

The embodiments of FIGS. 28 to 33 are based on a threaded engagement between the activation member and the cap. The embodiment shown in FIGS. 34 to 38 uses a bayonet type engagement between the activation member 18 and the cap 34. FIG. 34 is a perspective view of the front end of an activation member in which the aperture 161 in the front face 29 of the activation member 18 has four lugs 160 projecting into it. These lugs 160 engage with corresponding slots formed on the interior of a cap 34. A cross sectional perspective view of the cap for engagement with the activation member shown in FIG. 34 is shown in FIG. 35. Between the latches 162 formed in the cap 34 to engage the needle shield 40 there are four slots 164 corresponding to the four lugs 160 formed on the activation member. At the bottom of the slots 164 is an undercut with a recess 166 in which each lug 160 sits in order to prevent the activation member 18 moving to the activation position. FIG. 36 is a perspective cross sectional view of the cap of FIG. 35 engaged with the activation member of FIG. 34. The lugs 160 can be seen engaged in recess 166 so that, whilst the cap is abutting the housing 26, the activation element 18 cannot move rearwardly into the housing 26 to the activation position. The latches 162 are shown clearly engaging the removable needle shield 40.

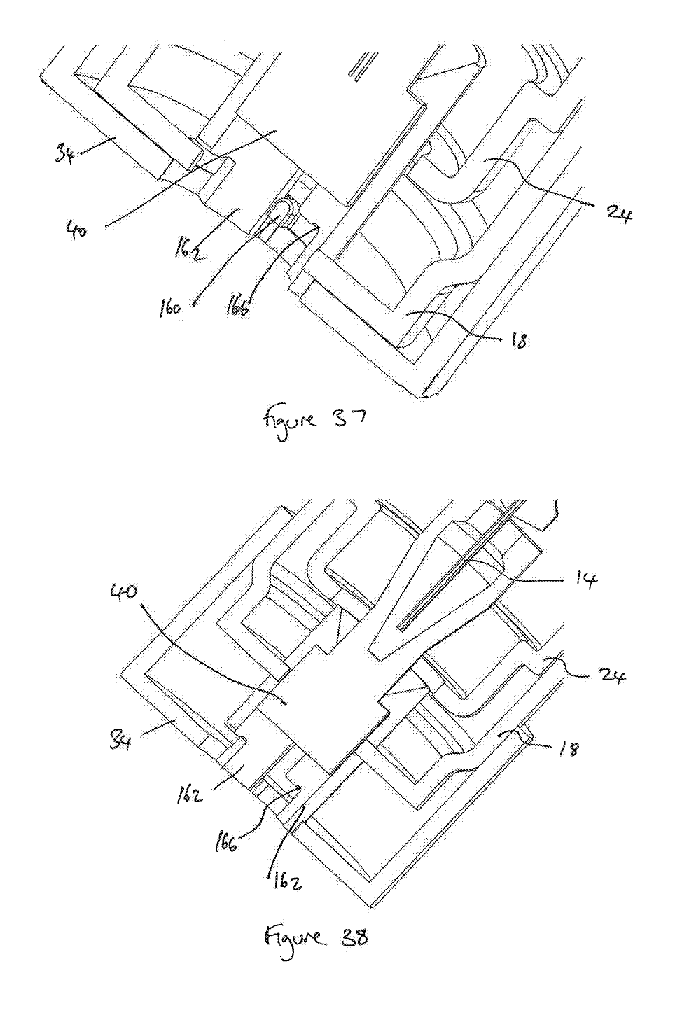

In order to remove the cap and allow the autoinjector to be activated, the cap 34 is first rotated relative to the activation member 18 to move the lug 160 out of the recess 166 to a position at the bottom of the slots 164. This position is shown in FIG. 37. The cap 34 can then be pulled with a longitudinal translational movement away from the housing 26 to remove the cap 34 and the needle shield 40, so that the device is ready for use. FIG. 38 shows the cap 34 during removal, as the needle shield 40 is pulled off the drug container. In this position the activation member is free to move back into the activation position.

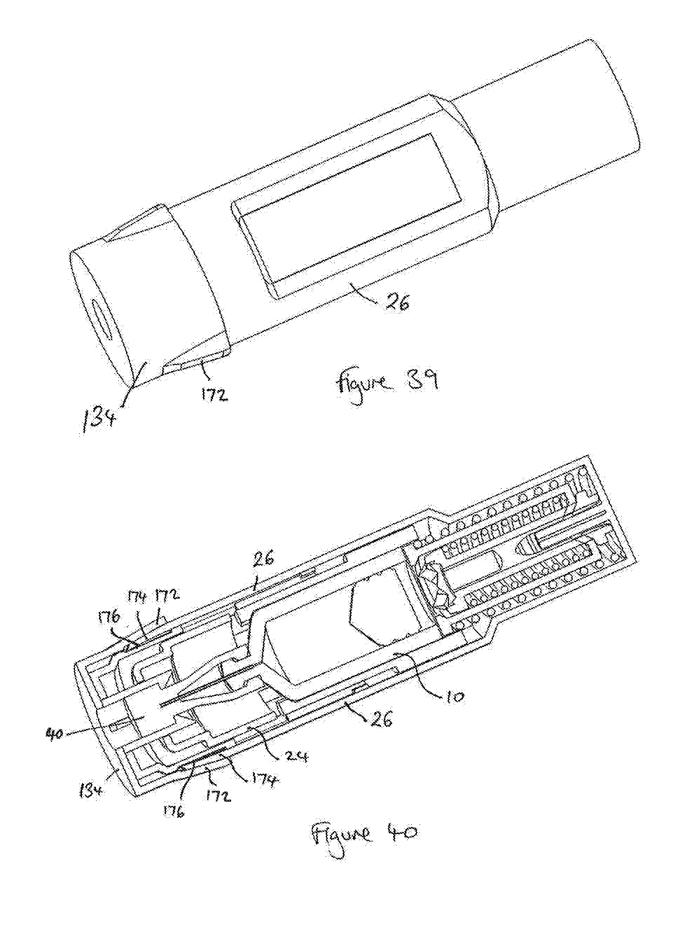

FIG. 39 illustrates a twelfth embodiment of the invention. FIG. 39 is a perspective view of an autoinjector of the type shown in FIG. 1 with a cap 134 placed over the activation element. FIG. 40 is a cross section of the autoinjector shown in FIG. 39. The housing 26 of the autoinjector includes a pair of flexible fingers 174 that extend into corresponding recesses 176 formed in the activation member. The flexible fingers 174 are retained in the recesses 176 by walls 172 of the cap 134 and thereby prevent rearward movement of the activation member 18 to the activation position. This is more clearly shown in FIG. 42. Once the cap 134 is removed from the housing 26, the flexible fingers 174 can deflect out of the recesses 176 to allow the activation member to move to the activation position.

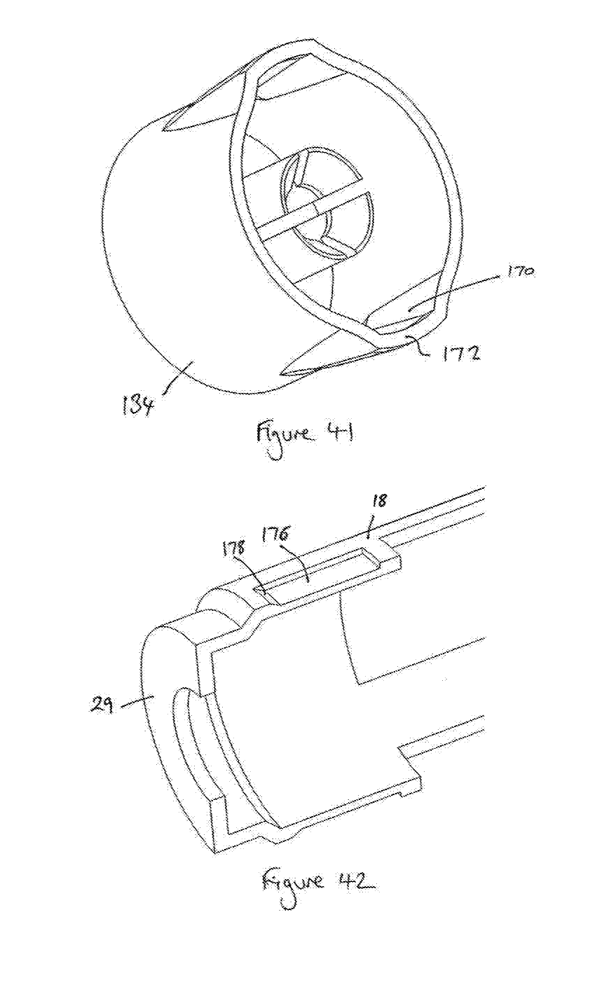

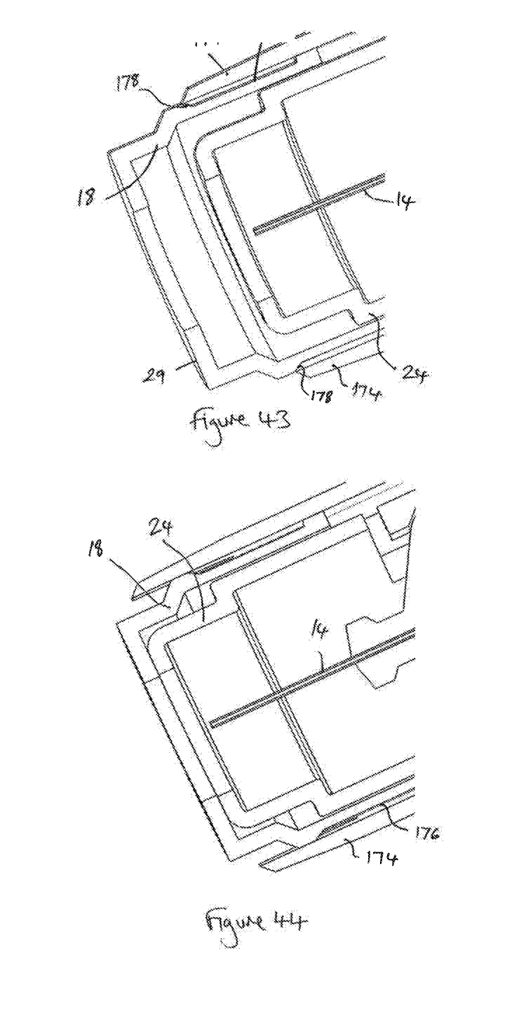

FIG. 41 is a perspective view of the cap 34 shown in FIGS. 39 and 40. The cap includes radially enlarged pockets 170 the walls 172 of which engage the flexible fingers 174 and retain them inside the recesses 176 formed in the activation member. FIG. 42 is a perspective cross sectional view of the activation member 18 and illustrates the recess 176 formed on the outer surface of the activation member 18. A front end of the recess 176 includes an angled or chamfered surface 178 over which a front end of the flexible finger 174 travels as the activation member 18 moves rearwardly to the activation position. This is illustrated in FIG. 43 which shows the flexible finger 174 riding on the surface 178 to deflect out of the cavity 176. FIG. 44 shows the activation element 18 moved to the activation position, with the flexible fingers 174 positioned on the outside of the activation member, clear of the recesses 176.

Although multiple separate embodiments have been described in the specification, it should be clear that features of one or more of the embodiments could be combined with the features of one or more of the other embodiments. For example, the cap may be fixed to the housing by a helical thread engagement while being engaged to the activation member by an intermediate component of the type shown in FIG. 16, 21 or 25. It is also possible to include in a single implementation more than one mechanism to lock the activation member against moving to the activation position.

Although the embodiments have all been described in relation to in particular autoinjector design, it should be clear that the invention is applicable to any drug delivery device which is activated by pressing an activation element against a surface.

1. An injector device comprising:
a housing;
a drug container positioned in the housing;
a drug delivery mechanism associated with the drug container, the drug delivery mechanism comprising a stored energy source;
an activation member coupled to the housing, the activation member configured to contact the injection site in use and movable relative to the housing to an activation position to activate the drug delivery mechanism; and
a removable cap coupled to the activation member and to the housing, wherein in a first position a first portion of the housing engages the activation member to prevent the activation member from moving into the activation position, and wherein a portion of the cap engages the housing to retain the first portion of the housing in the first position such that the activation member is prevented from moving into the activation position until the cap is uncoupled from the activation member.

2. An injector device according to claim 1, wherein the first portion of the housing is flexible and the cap engages the housing to prevent the first portion of the housing moving out of a first position engaging the activation member, wherein on removal of the cap, the first portion of housing is able to move out of the first position to allow the activation member to move to the activation position.

3. An injector according to claim 2, wherein the cap engages the housing to deflect the first portion of the housing into the first position.

4. An injector device according to claim 1, wherein in the first position the first portion of the housing engages a slot or recess in the activation member.

5. An injector device according to claim 1, wherein the activation member includes a cam surface configured to allow the activation member to move past the first portion of the housing after the cap has been removed from the housing.

6. An injector device according to claim 1, wherein a portion of the housing engages the cap to retain the cap.

7. An injector device according to claim 1, wherein the cap is coupled to an exterior surface of the activation member.

8. An injector device according to claim 1, wherein the activation member is configured to move telescopically with respect to the housing.

9. An injector device according to claim 1, wherein a rear end of the activation member engages with one or more parts of the delivery mechanism or with a locking arrangement for the delivery mechanism.

10. An injector device according to claim 1, wherein the first portion of the housing comprises a pair of flexible fingers.

11. An injector device according to claim 1, wherein the injector device is an autoinjector.

12. An injector according to claim 1, wherein in the first portion of the housing comprises at least one flexible finger that, in the first position, extends into a corresponding recess formed in the activation member.

13. An injector according to claim 12, wherein the removable cap engages an exterior surface of the flexible finger to retain the flexible finger in the recess formed in the activation member to thereby prevent the activation member moving into the activation position.

14. An injector device according to claim 12, wherein the removable cap comprises a radially enlarged pocket that engages the flexible finger to retain it in the first position.

15. An injector device according to claim 4, wherein the slot or recess includes a cam surface configured to allow the activation member to move past the first portion of the housing after the cap has been removed from the housing.

16. An autoinjector comprising:
a housing;
a drug container positioned in the housing;
a drug delivery mechanism associated with the drug container, the drug delivery mechanism comprising a stored energy source;
an activation member coupled to the housing, the activation member configured to contact the injection site in use and movable distally relative to the housing, within the housing, to an activation position to activate the drug delivery mechanism; and
a removable cap coupled to an exterior of the activation member and to the exterior of the housing, wherein in a first position a first resilient portion of the housing engages a recess in the activation member to prevent the activation member from moving distally into the activation position, and wherein a portion of the cap engages the housing to retain the first resilient portion of the housing in the recess such that the activation member is prevented from moving into the activation position until the cap is uncoupled from the activation member.

17. An autoinjector according to claim 16, wherein in the first resilient portion of the housing comprises at least one flexible finger that, in the first position, extends into a corresponding recess formed in the activation member.

18. An autoinjector comprising:
a housing;
a drug container positioned in the housing;
a drug delivery mechanism associated with the drug container, the drug delivery mechanism comprising a stored energy source;
an activation member coupled to the housing, the activation member configured to contact the injection site in use and movable distally relative to the housing, within the housing, to an activation position to activate the drug delivery mechanism; and
a removable cap coupled to an exterior of the activation member and to the exterior of the housing, wherein in a first position a pair of flexible fingers formed on the housing engage corresponding recesses in the activation member to prevent the activation member from moving distally into the activation position, and wherein a portion of the cap engages the housing to retain the flexible fingers on the housing in the recesses such that the activation member is prevented from moving into the activation position until the cap is uncoupled from the activation member.
